(19) United States
(12) Patent Application Publication
(10) Pub. No.: US 2005/0014247 A1
(43) Pub. Date: Jan. 20, 2005

(54) Method and machine for ex situ production of low and medium intergration biochip networks

(76) Inventors: Michel Cabrera, Lyon (FR); Mehdi Jaber, Villeurbanne (FR); Jean-Pierre Clarec, Lyon (FR); Jerome Broutin, Lyon (FR); Eliane Souteyrand, Chambon sur Cisse (FR); Jean-Rene Martin, Lozanne (FR)

Correspondence Address: MORGAN LEWIS & BOCKIUS LLP, 1111 PENNSYLVANIA AVENUE NW, WASHINGTON, DC, 20004, US

(21) Appl. No.: 10/483,382

(22) PCT Filed: Jul. 10, 2002
(86) PCT No.: PCT/FR02/02427

(30) Foreign Application Priority Data
Jul. 10, 2001 (FR) 01/09179

(51) Int. Cl. B41J 29/38; C12M 1/34

(52) U.S. Cl. 435/287.2; 347/10

(57) ABSTRACT

A method of ex situ fabrication of at least one biochip, the method being of the type consisting in projecting onto a substrate a microvolume of reagent comprising at least one probe diluted in a suitable solvent so as to form, after the solvent has been eliminated, a spot comprising said probe, the method consisting in using a microprojection device comprising at least one tank in which the reagent is stored, and at least one source of gas under pressure put into communication with the tank, and in projecting the microvolume of reagent through an ejection nozzle under drive from the pressure exerted by the gas on the reagent.

The invention relates to method and a machine for ex situ fabrication of low and medium integration biochip arrays.

### BACKGROUND OF THE INVENTION

In the field of biotechnologies, biochips, and in particular DNA chips, are presented as being completely innovative tools capable of revolutionizing experimental approaches to molecular biology. The advantage of a biochip lies in its ability to identify target biomolecules in parallel with several tens to several millions of probes of different compositions on the same solid structure (glass, silicon, polymer, etc.), the probes being secured by implementing an affinity recognition process between the probes and the chip.

By way of example, a DNA chip is a device designed to enable monocatenary DNA strands to be identified by using the hybridizing process implemented between the strand(s) of the target DNA to be recognized and one of the known-sequence oligonucleotides probes fixed in a defined zone on a solid support, and then by identifying the hybridized zone and thus the oligonucleotide sequence it supports, it is possible to determine the sequence of the target DNA strand.

Depending on the degree of integration of biochips, the following can be distinguished:
low integration chips having up to a few hundred probes;
medium integration chips possessing several hundreds to several thousands of probes; and
high integration chips having more than ten thousand probes;
it being understood that these various types of chips are not used for the same purposes.

The main application for low integration biochips in the field of diagnosis. They are therefore intended for mass production at relatively low cost. Their use must lead to a result that is simple and that can be interpreted rapidly. Their fabrication advantageously makes use of direct immobilization of probes obtained from natural or presynthesized extracts that have previously been checked and purified. That technique guarantees probe purity and thus better reliability for the chips that are prepared.

Medium integration biochips are usually used for very precise studies, often for the purpose of developing low integration chips, for example DNA chips for studying the mutation of a gene or transcryptome. They therefore need to be produced in smaller quantities than low integration chips and their production requires tooling that is highly adaptable. For biochips at the small end of medium integration, immobilization can be used, whereas for biochips at the large end of medium integration it is appropriate to make use of in situ synthesis directly on the support of the chip.

High integration biochips are used for studies that are very complex and expensive. The preparation of such chips can be envisaged only in terms of in situ synthesis. For it to be possible to interpret reliably the large quantity of information obtained on a chip, associated with the large number of probes, and thus the large number of possible solutions, a large number of experimental operations are required for validating the affinity recognition process, e.g. hybridization/denaturing for DNA, and also a large amount of mathematical processing. Such very lengthy development leads to high costs, which limits applications to studies having high added value, for example studying the side effects of medicines.

Numerous methods of fabricating biochips are known, and they can be classified in two categories, namely in situ methods of fabrication which consist in simultaneously synthesizing the probes base by base on a substrate, and ex situ methods of fabrication which are based on producing the set of probes and then fixing the probes to the substrate.

The invention lies within ex situ methods of fabrication where the probes are fabricated using methods that are known per se. Ex situ methods of fabrication can be classified by the method used for putting the probe into contact with the substrate: either the solution containing the probe is put into contact with the entire surface of the substrate, with fixing then taking place in a zone that is addressed selectively, e.g. by electropolymerization, or else the probes are deposited locally by physical contact of the deposition means with the substrate, or indeed by microprojection with no physical contact with the substrate. Ex situ methods of fabrication can also be classified depending on the method of bonding the probes to the substrate. The various possible bonding methods are based on adsorption of the probe on the substrate, or on bonding by biological affinity, e.g. of the biotin/streptavidin type, or by bonding by electropolymerization, or, advantageously, by forming a covalent chemical bond. The method of the invention is compatible with all of those fixing methods.

In known methods of ex situ fabrication with probes being bonded to the substrate by microprojection, piezoelectric or thermal effect type devices are used, where those two techniques have been developed for ink jet printers.

Piezoelectric type microprojection devices are devices comprising a probe tank which is fed by capillarity and/or under the effect of a very small positive or negative pressure relative to the outside, i.e. a difference of millibar order. The tank has an orifice that is always open, so problems arise of probe conservation due to possible evaporation and contact with the atmosphere. Furthermore, microprojection using a device of this type is limited to a maximum volume of the order of 4 picoliters (pl) to 30 pl for a tank having a volume of milliliter (ml) order, which means that operations of decontamination by replacing the probe-containing solution with a cleaning liquid are not easy, nor are emptying operations, given the small flow rate that can be achieved. Finally, piezoelectric type microprojection devices require control devices that are complex and difficult to develop.

### OBJECTS AND SUMMARY OF THE INVENTION

An object of the invention is to devise another ex situ technique for projecting drops of reagent onto a substrate, the novel technique also being well adapted to fabricating biochips having several hundreds to several thousands of probes, with fabrication taking place at high speed and with fabrication costs of a few euro cents to a few euros per chip, thus making it possible to satisfy the needs of industry concerning mass production of low and medium integration chips.

To this end, the invention provides a method of ex situ fabrication of at least one biochip, the method being of the type which consists in projecting onto a substrate a microvolume of reagent comprising at least one probe diluted in a suitable solvent so as form, after elimination of the solvent, a spot containing said probe, which method consists in using a microprojection device having at least one tank in which the reagent is stored, and at least one source of gas under pressure put into communication with the tank, and in projecting the microvolume of the reagent through an ejection nozzle under drive from the pressure exerted by the gas on the reagent.

To this end, the method consists in associating the microprojection device with an actuator interposed between the tank and the ejection nozzle, and in controlling the actuator to occupy an “open” state for a determined length of time in order to put the tank into direct communication with the ejection nozzle, thereby projecting the microvolume of reagent under drive from the pressure of the gas present in the tank.

In general, the method consists in continuously maintaining the reagent stored in the tank under pressure, and in causing the actuator to take up a “closed” state in order to isolate the tank under pressure while waiting to eject a microvolume of reagent, thus making it possible to avoid the reagent evaporating and enabling it to be conserved in a medium that is inert.

Advantageously, the method consists in using a battery of independent microprojection devices to form a plurality of spots on at least one substrate, and in configuring the battery of microprojection devices in a matrix having a plurality of rows, in making each row of the matrix in modular form, and in putting all of the tanks of a row of microprojection devices into communication with the same source of gas under pressure.

In general, the method consists in placing at least one substrate on a plate, and in imparting relative displacement between the plate and the battery of microprojection devices so as to form a plurality of spots on the substrate using a firing or ejection process that is sequential or on-the-fly.

Furthermore, with a large number of microprojection devices, it is possible in a single pass to form a plurality of spots on at least one substrate, each tank of the microprojection devices containing only a single type of probe.

The method also consists in calibrating the microvolumes of reagent projected onto the substrate by controlling the direction in which reagent is projected through each nozzle, and in also performing quality control.

In practice, the method consists in placing the ejection nozzles at a distance from the substrate of about 0.1 millimeters (mm) to 10 mm, preferably about one millimeter, and in projecting a microvolume of reagent in the form of a microdroplet having a volume of the order of a few nanoliters (nl).

The invention also provides a machine for implementing the above-defined method, which machine comprises at least one microprojection device for projecting at least one microdroplet of reagent onto at least one substrate, the device comprising at least:
a tank in which the reagent for projection is stored;
at least one source of gas under pressure put into communication with the tank via an inlet tube;
an actuator connected to the tank by an outlet tube having one dipping into the tank; and
an ejection nozzle mounted at the outlet from the actuator and communicating directly with the tank when the actuator is in an “open” state under the control of a control circuit.

Advantageously, the actuator of each microprojection device is constituted by an electrically controlled valve.

In an advantageous embodiment, the machine comprises a battery of microprojection devices which are independent from one another, and the battery of microprojection devices is configured as a matrix comprising a plurality of rows.

Advantageously, each row of the battery of microprojection devices forms a module whose structure may comprise at least:
a first support block in the form of a bar for supporting all of the tanks of the module and for providing the fluid connections needed to put the reagent stored in the tanks under pressure; and
a second support block for supporting the actuators and the ejection nozzles of the microprojection devices of the module.

In an embodiment, the first support block is pierced by a main through longitudinal channel having one end connected to a source of gas under pressure, and it is also pierced by a set of secondary transverse channels opening out into the main channel and into all of the tanks, with there being one secondary channel per tank, thereby using a single source of gas under pressure for all of the tanks of the module, so as to reduce the number of connections.

Furthermore, the support block is also pierced by through transverse orifices with outlet tubes passing therethrough connecting the tanks to the actuators, which orifices can be disposed in a staggered configuration to reduce the size of the module.

Advantageously, each outlet tube is made up of two segments which are connected together at a transverse orifice of the first support block by means of a quick coupling.

Advantageously, the structure of each module is removably mounted on the frame of the machine so as to facilitate operations of mounting and removing modules.

In general, the machine also comprises a plate for supporting at least one substrate, and means for imparting relative displacement between the plate and the battery of microprojection devices.

In an embodiment, the battery of microprojection devices is stationary and the plate moves under the control of a crossed XY movement device by means of two motors.

The method and the machine for ex situ fabrication in accordance with the invention enable mass production to be implemented, which was not possible in the past. In machines for implementing mechanical microdeposition methods, often referred to as “spotters” or as “arrayers”, pipette matrices are used comprising four to 32 elements, whereas the number of wells is generally 128 or 384. It is thus not possible to deposit all of the probes in a single pass, and after each pass it is also necessary to decontaminate and rinse the pipettes. Similarly, in machines of the piezoelectric type or the thermal effect type for implementing microprojection methods, it is difficult to envisage fabricating more than a few microprojection means, which implies frequent cleaning and emptying operations.

Using positive pressure for microprojecting reagents makes it possible to design a machine of operation that is stable. The rate at which the reagents stored in the tanks are used up has little influence on the rate at which the microprojection devices operate, since their leaktightness prevents the solvent from evaporating, i.e. the concentration of probes does not vary, and it is possible to reuse probes that have not been used and that have been conserved in their respective tanks, without suffering any loss of quality, since there is no contact with a gas or a reagent that might spoil the properties of the probes which are conserved throughout use in an inert medium. Furthermore, by adjusting the gas pressure, it is possible to work with probes of high viscosity or different viscosities, and this is particularly advantageous, for example with DNAc probes.

The method and the machine of the invention for ex situ fabrication also presents numerous advantages for mass production, and in particular:
small consumption of reagents given that the microprojection device constituted by a micro solenoid valve with an integrated nozzle ejects drops having a volume that is typically 10 nl, whereas the “working”, i.e. projectable volume of probe in the tank is of the order of 1 ml, thus making it possible to project about 100,000 drops and to fabricate about 100,000 chips in a continuous mass production fabrication process;
low cost of fabrication for the chips because of the small quantity of biological material used by the chips as a whole; by way of example, the cost of a tank of oligonucleotide probes containing 25 nucleotides is about 30 euros, which compared with manufacturing a volume of 100,000 chips leads to a cost of 0.03 euro cents per spot and per chip, which means that in the cost of manufacturing a chip having 256 probes, the quantity represented by the probes in the fabrication cost is less than 0.15 euros;
implementation is simple;
the microprojection method is compatible with a large number of solvents whether pure or mixed, having a variety of physicochemical properties (surface tension, viscosity, evaporation rate), such as, for example: acetonitrile; dimethylformamide (DMF); dimethylsulfoxide (DMSO); water; and aqueous solutions such as TRIS, phosphate buffer solution (PBS), where this solvent is particularly advantageous for the following reasons: it is innocuous; it presents good solubility of biomolecules, making it possible to obtain spots that are uniform; its surface tension is adapted to activated substrates, thus making it possible to obtain spots of small size; it eliminates solvent in controlled manner, e.g. for a 250 μm spot constituted by projecting 10 nl of probe in PBS, the solvent is eliminated in 60 seconds (s) at 21° C. and in 15 s at 40° C.; and
production is flexible, given that it is possible, for example, to mass produce a chip having 1024 probes by using four devices each having 256 projection means, it being understood that for small series at laboratory scale, it is possible to fraction production and obtain 1024 probes with the 256 tank machine by changing tanks four times, with an intermediate washing operation, and by offsetting the microprojection onto the substrate.

In general, the use of biochips opens up huge perspectives in a very wide variety of fields, such as, for example:
fundamental research: genome sequencing, studying the expression and the functions of genes, mutation research, studying the biodiversity of species (animals, plants, bacteria);
the medical field: developing diagnostic tests for infectious and genetic diseases, assistance in tracking clinical trials, adapting therapy;
pharmaco-genomics: treatment toxicity and identifying side effects, resistance to antiviral drugs, searching for new molecules;
the environment: monitoring pollution by bacterial analysis of water, air, and soil; and
food business: bacteriological monitoring, identifying genetically modified organisms (GMOs), etc.

In general, probes can be secured via a layer of bonding molecules having at one end a function for fixing them to the substrate and at the other end a function, possibly a protected function, that serves to avoid reacting with the support. This function can then be used to fix a probe by means of a covalent bond, once the function has been deprotected and activated.

The method of fabrication may be based on covalent bonding of probes on the substrate, which may be implemented on the basis of the following chemistry:
the substrate is functionalized with a silane whose free end carries a methyl ester function;
the silane is deprotected, e.g. under the action of concentrated hydrochloric acid, giving carboxlyic acid, and it is then activated, e.g. under the action of N-hydroxysuccinimidine in a suitable solvent such as tetrahydrofuran (THF), giving an activated ester; and
the probe provided with an amine function is deposited on the substrate so as to form a covalent amide bond between the probe and the bonding molecule on the substrate.

Naturally, it is possible to use other chemical systems that may be based, for example, on the following groups: thiol, epoxy, carboxylic acid (fixed to the probe end), primary amine (fixed to the surface end), . . . .

In general, the invention enables arrays to be fabricated in a wide variety of dimensions, degrees of integration (number of probes per substrate), densities (number of probes per unit of area), and compositions, i.e. with probes constituted by oligonucleotides, DNA, DNAc, RNA, peptide nucleic acids (PNA), enzymes, immunoproteins, proteins, cells, and in general biological substances on any kind of support, and in particular supports made of silica, silicon, metal, or polymer, having an area of 1 square millimeter (mm²) to 10,000 mm². The area of a spot lies within a circle having a diameter of 1 micrometer (μm) to 500 μm. The number of spots can lie in the range 1 to 10,000 per substrate. Probes of different chemical species can be used simultaneously on the same substrate, e.g. oligonucleotides, peptide nucleic acids (PNA), and proteins.

### BRIEF DESCRIPTION OF THE DRAWINGS

Other advantages, characteristics, and details of the invention appear from the additional description below given with reference to the accompanying drawings that are given purely by way of example, and in which:

FIG. 1 is a simplified view of a device for microprojecting a microvolume of reagent onto a substrate;

FIG. 2 is an example of a control circuit for the microprojection device shown in FIG. 1;

FIG. 3 is a diagrammatic view to illustrate the concept of a battery of microprojection devices;

FIG. 4 is a perspective view to give an overall example of a modular machine fitted with a battery of 256 microprojection devices;

FIG. 5 is a perspective view of a module of the machine shown in FIG. 4;

FIG. 6 is a view looking along arrow VI in FIG. 5;

FIG. 7 is a section view on line VII-VII of FIG. 6;

FIG. 8 is a view similar to the view of FIG. 7;

FIG. 9 is a diagrammatic view showing the principle on which the machine shown in FIGS. 4 to 7 is controlled;

FIGS. 10 and 11 are diagrams showing 32 chips being fabricated with a machine of the type shown in FIGS. 4 to 7;

FIGS. 12 and 13 show a substrate on which spots are formed, both before and after a process for automatically correcting their positions on the substrate;

FIG. 14 is a perspective view of the machine fitted with a display system to monitor the formation of spots;

FIG. 15 is a side view of FIG. 4; and

FIGS. 16 and 17 are fragmentary views similar to those of FIGS. 14 and 15 and showing a variant mount for the display system.

### MORE DETAILED DESCRIPTION

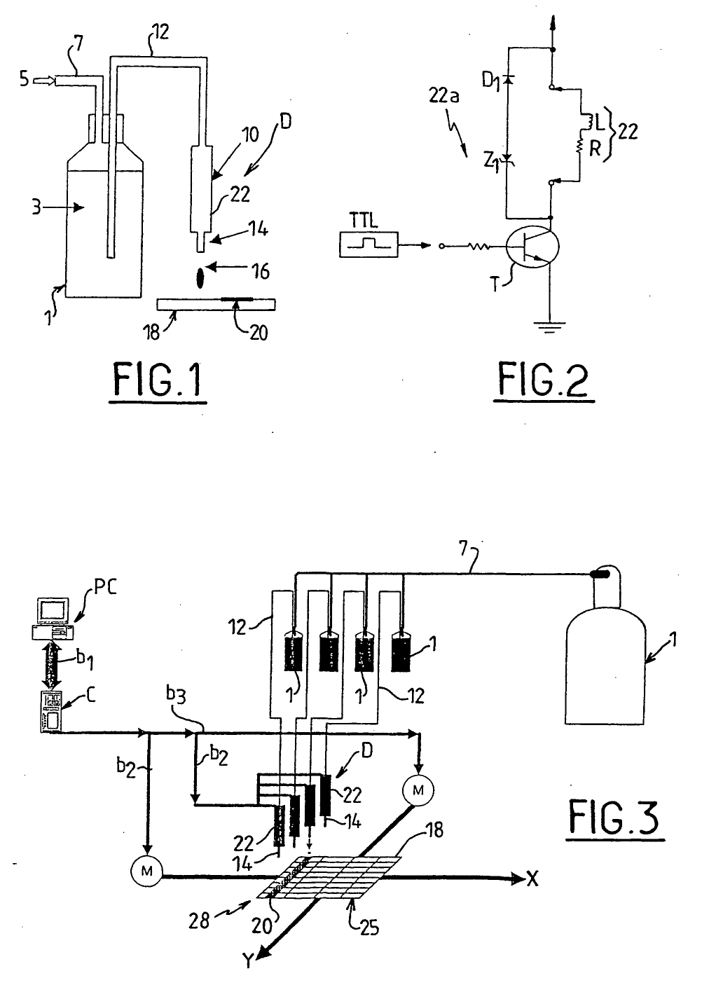

As shown in FIG. 1, a microprojection device of the invention comprises at least:
a tank 1 storing a reagent 3 in liquid form that is constituted by at least one probe diluted in a suitable solvent;
a source 5 of gas under pressure which is connected to the tank 1 via an inlet tube 7, the gas used being an inert gas such as helium, for example;
an actuator 10 which is connected to the tank 1 by an outlet tube 12 which dips into the reagent 3; and
an ejection nozzle 14 which is mounted at the outlet of the actuator 10.

In general, the function of the actuator 10 is to cause a microvolume of reagent 3 to be projected through the nozzle 14. The actuator 10 may occupy at least two states, respectively an open state and a closed state. By occupying the open state for a determined length of time, the actuator 10 allows reagent 3 to pass into the nozzle 14 under drive from the pressure of the gas that exists in the tank 1, and providing the pressure is sufficient, this causes a microvolume to be ejected in the form of a microdroplet 16 which becomes deposited on the activated surface of a substrate 18 so as to form a spot 20 once the solvent has been eliminated.

The actuator 10 is advantageously constituted by a micro solenoid valve 22. A micro solenoid valve presents a dead volume that is very small and it is less sensitive to variations in reagent viscosity than is an actuator controlled by a piezoelectric element, for example. The valve 22 is controlled by a control circuit 22a of the type shown diagrammatically in FIG. 2, where from the electrical point of view the valve 22 presents equivalent resistance R and inductance L connected in series. The control circuit 22a may comprise no more than a transistor T whose base is controlled by transistor transistor logic (TTL) type control signals, for example, and whose emitter is connected to ground. The RL equivalent circuit of the valve 22 is connected in series between the collector of the transistor T and a power supply voltage. A series connection of a diode D₁ and a zener diode Z₁ is connected in parallel with the RL circuit to reduce the parasitic effect of oscillations in the inductance L, with the diode Z₁ limiting voltage, while the diode D₁ prevents reverse current flow.

In general, the volume of reagent which is ejected by the nozzle 14 and the extent to which it spreads on the substrate depend on various factors, and in particular on:
the material, the nature, and the shape of the hydraulic connections;
head losses in the device;
the nature and the shape of the ejection nozzle;
the reagent stored in the tank; and
the length of time the micro solenoid valve is open, the pressure in the tank in which the reagent is stored, the distance between the ejection nozzle and the substrate, . . . .

By way of example, the control circuit 22a is designed so as to be capable of opening and closing the valve 22 quickly in a time of about 0.01 milliseconds (ms) to 1 s in order to eject a volume of reagent lying in the range about 0.1 nm to 1000 nl, in the presence of positive pressure in the tank 1 that is about 0.05 bars to 1 bar.

In general, a battery of microprojection devices is used to project microdroplets of reagent (probes) diluted in solvent, in particular onto substrates that are plane, in order to proceed with a chemical reaction whereby the probes are bonded to the substrate once the solvent has evaporated, giving rise to a dry substrate having a plurality of probes grafted thereon. Advantageously, a battery of independent microprojection devices is used so as to be able to make a plurality of biochips simultaneously, it being understood that each biochip may optionally be different concerning the disposition of the probes on the biochip, since there is no relationship between the disposition of the microprojection devices and the disposition of the spots on the biochip. Furthermore, the tanks of the microprojection devices can be changed independently from one another, and one microprojection device can be allocated to each probe so as to avoid problem of contamination between probes within the microprojection devices themselves.

With the basic configuration shown in FIG. 3, a battery of independent microprojection devices D are provided, there being sixteen such devices disposed in a row. Advantageously, in order to reduce the number of connections, a single source 5 of gas under pressure is used to feed all of the tanks 1, which also makes it possible to operate under steady conditions and to prevent solvent from evaporating. In general, a plurality of activated substrates 18 can be placed on a plate 25 and provision is made to impart relative movement between the battery of microprojection devices D and the plate 25 in order to form the spots 20 on the substrates 18, it being understood that the plate 25 may be of dimensions suitable for carrying one to 10,000 substrates, and typically about 100 substrates for performing mass production. By way of example, the battery of microprojection devices may deliver fluid in quantities of 1 nl to 30 nl, the valves being opened for durations of about 0.1 ms to 2 ms and the fluid being under a pressure of 0.01 bars to 0.5 bars, so as to form spots having a diameter of about 100 μm to 1000 μm, with spacing between spots of about 50 μm to 500 μm.

In the example shown in FIG. 3, the battery of microprojection devices D is stationary while the plate 25 is mounted on a motor-driven device 28 for imparting crossed X and Y movements under the control of two motors M. Advantageously, the micro solenoid valves are in alignment on a row parallel to the Y displacement axis of the plate 25.

The valves 22 are controlled by a control member, e.g. a personal computer (PC), associated with an electronics card C connected to the PC via a control, address, and data bus b₁, and to the microprojection devices D via a bus b₂, and to the motors M via a bus b₃. The controlling PC serves to synchronize the opening of the valves 22 with the position of the substrate-carrying plate 25 in such a manner that the microdroplets are delivered at precise and desired locations on the substrates 18.

Thus, in order to fabricate a batch of chips, it suffices to place the plate 25 quickly and successively beneath the ejection nozzles 14 of the micro solenoid valves 22 while activating projection of microdroplets of reagent as the substrates 18 move past. Immediately after being projected, the solvent is eliminated so as to form spots 20 comprising probes fixed to the substrates 18 by covalent bonds, in conventional manner.

Furthermore, it is possible to project the same microdroplet of reagent several times over onto the substrates 18, onto the same spot 20 or more advantageously onto a plurality of spots, so as to constitute a pattern suitable for making it easier to read the chips, thereby possibly improving the reliability of diagnosis by means of a redundancy effect.

Specifically, there is no possibility of contamination whether between the microprojection devices D, or between the devices and the substrates 18, given that the spacing between the ejection nozzles 14 and the substrate 18 is about 0.1 mm to 10 mm, and preferably about 1 mm, where said spacing is considerably greater than the diameter of the microdroplets of reagent that are projected.

Starting from this basic configuration, it is possible to design a machine. 30 that is capable of operating at high speed since the reagent is projected without the machine coming into contact with the substrate. The speed of operation is limited essentially by the speed at which the plate 25 can be displaced which may lie in the range about 1 centimeter per second (cm/s) using direct current (DC) motors M, to about 10 meters per second (m/s) with magnetic linear motors.

Advantageously, the battery of microprojection devices D is arranged in the form of a matrix comprising a plurality of rows, and each row is designed in modular and removable manner.

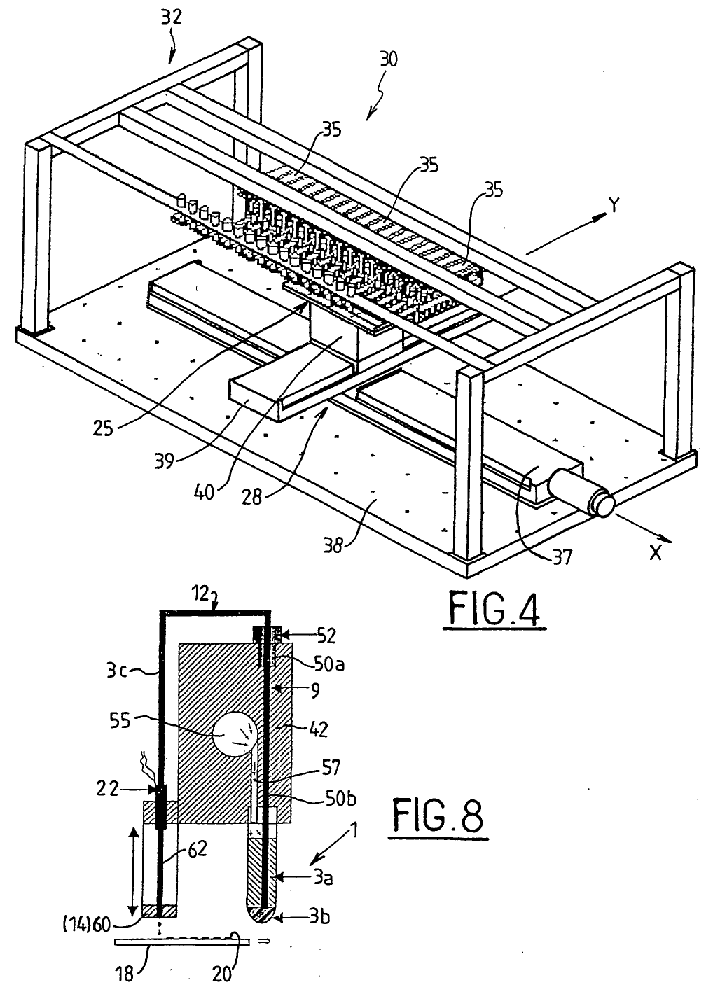

A machine 30 having a battery of 256 microprojection devices D is shown diagrammatically in FIG. 4. The machine 30 presents a frame 32 including a crossbar which supports 16 mutually parallel double rails 34, with sixteen modules 35 mounted therealong each supporting sixteen tanks 1 with associated micro solenoid valves 22. The activated substrates 18 are placed on a plate 25 which is movable by means of a motor-driven device 28 delivering crossed XY movements of large amplitude and large displacement speed.

The motor-driven device 28 is constituted by a longitudinal guide block 37 (X axis) fixed on a baseplate 38 of the frame 32 by means of a transverse guide block 39 (Y axis) mounted to slide on the longitudinal guide block 37, and by a support block 40 slidably mounted on the transverse guide block 39 and supporting the plate 25.

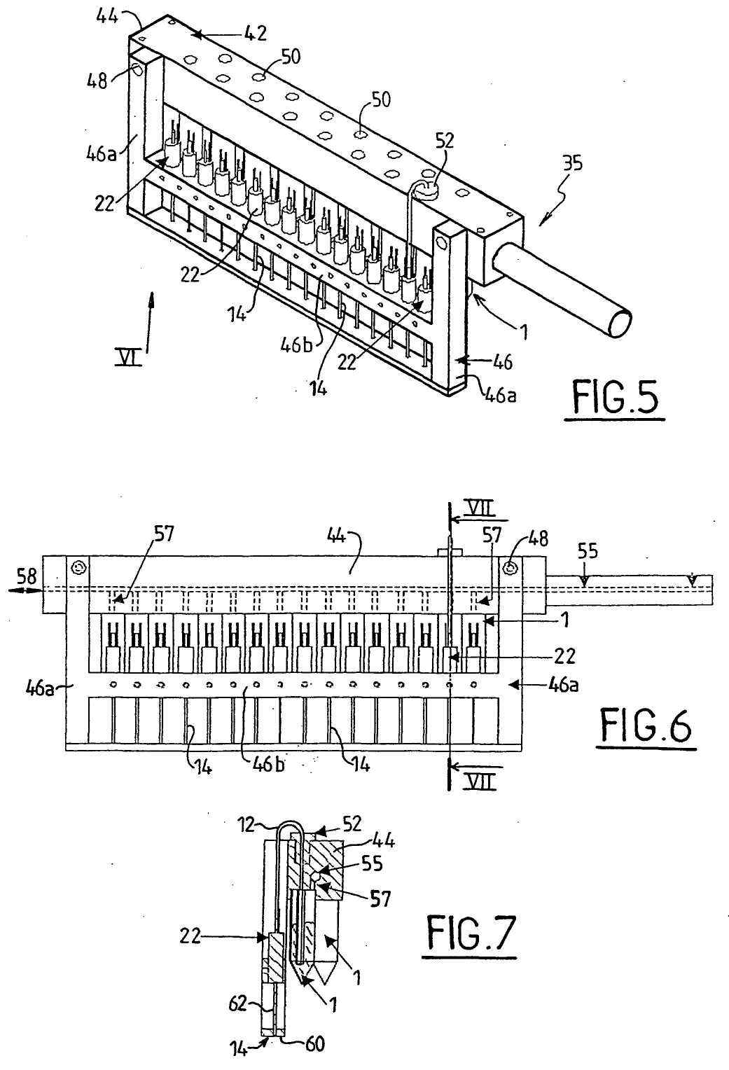

In an embodiment shown in FIGS. 5 to 7, each module 35 comprises a structure 42 which is constituted as follows (FIGS. 5 and 6):
a first support block 44 in the form of a bar for supporting sixteen tanks 1 and also for providing the fluid connections that are needed to put the reagent 3 stored in the tanks 1 under pressure; and
a second support block 46 of H-shape having two parallel uprights 46a interconnected by a horizontal crossbar 46b which supports sixteen micro solenoid valves 22.

The top ends of the two uprights 46a are connected to the first support block 44 near its two ends by fastening means 48, it being possible for the tanks 1 to be situated at a height above that of the ejection nozzles 14 of the associated valves 22.

The sixteen valves 22 are in alignment along the crossbar 46b so as to be substantially in register with the sixteen associated tanks 1, it being understood that the tanks may advantageously be disposed in a staggered configuration (FIG. 5) so as to reduce the length of the module 35, thereby limiting the Y displacements of the plate 25, and increasing the speed of operation of the machine 30.

For this purpose, the first support block 44 is pierced by sixteen through openings 50 disposed in a staggered configuration. Each opening 50 presents a portion 50a which is extended by a portion 50b. Each tank 1 is connected to the associated micro solenoid valve 22 via an outlet tube 12 made of polytetrafluoroethylene (PTFE) for example, which is in the form of an upside-down U-shape with one branch dipping into the corresponding tank 1, and with the other branch passing through an opening 50 for connection to the associated valve 22.

Each outlet tube 12 may be fixed to the first support block 44 via a quick coupling 52 of the screw type or the like which is mounted in the portion 50a of each opening 50. Advantageously, each outlet tube 12 is split into two segments which are connected together in leaktight manner via a screw coupling 52, thus enabling the tank 1 to be connected and disconnected quickly with the associated valve 22, thereby facilitating assembly and maintenance operations.

With reference to FIGS. 6 and 7, the first support block 44 in the form of a bar is also pierced by a longitudinal through channel or main channel 55. One end of this channel 55 is connected to a source 5 of gas under pressure by a self-sealing type coupling so as to deliver positive pressure simultaneously to the reagents 3 stored in all of the tanks 1 via transverse channels 57 that are likewise pierced in the first support block 44, all of which open out into the main channel 55 and each of which opens out into a respective tank 1. The opposite end of the main channel 55 is connected to a purge valve 58.

It is also possible to connect the modules 35 to sources of gas at different pressures.

The ejection nozzles 14 are aligned and mounted in such a manner as to be situated facing the substrates 18 and at a distance therefrom of millimeter order. Each ejection nozzle 14 may be constituted merely by a tube of PTFE of appropriate shape which is connected to the outlet of the associated micro solenoid valve 22.

Advantageously, as shown in FIG. 7, each ejection nozzle 14 is constituted by a part 60 such as a substrate of sapphire, ruby, silicon, . . . , or by a part made of ceramic, or stainless steel, which is pierced by an ejection orifice having a diameter of about 1 μm to 100 μm, typically 75 μm, and connected to the outlet of the micro solenoid valve 22 by a connection tube 62.

In addition, it is particularly advantageous to place the ejection nozzles 14 as close as possible to the associated micro solenoid valves 22 by minimizing the lengths of the connection tubes 62 so as to transmit the inlet pressure of the modules 35 with as little head loss as possible to the volume of reagent that is to be projected. Such a disposition makes it possible to use a lower inlet pressure, to obtain a better distribution within the valves 22, and to reduce the volume that is projected. Furthermore, it enables priming stages (as described below) to be reduced.

In normal operation of a module 35, the purge valve 58 is closed, as are all of the micro solenoid valves 22. In order to prime a module 35 prior to fabricating chips, each valve 22 is opened for a determined length of time so that the pressure of gas in the tank 1 expels the air from the outlet tube 12 and the connections, thereby causing the reagent to advance until it is flush with the corresponding ejection nozzle 14.

In practice, a plurality of primary projection operations are performed with a bowl taking the place of the plate 25. Since the reagent remains under constant pressure, it suffices to operate the micro solenoid valve 22 to cause a microdroplet of reagent to be projected.

During chip fabrication, and if the waiting time between two successive shots from the same microprojection device D is too long, then a mini-priming stage needs to be performed by proceeding with a few microprojection operations onto a trash zone of the plate 25. This mini-priming operation serves to compensate for any evaporation of reagent from the ejection nozzle 14.

The modular design of the machine 30 makes it possible to remove one module 35 independently from the others by sliding it along the rails 34, thus making it possible to change the tanks 1 and to maintain the micro solenoid valves 22.

In general, the chips are made on substrates of glass, silicon, metal, metal oxide, or polymer, with such homogeneous or heterogeneous substrates being previously functionalized and possibly being of different shapes.

A substrate in widespread use is a microscope slide having dimensions of 76 mm×26 mm, for example, since its cost price is low.

Furthermore, reducing the area of chips makes it possible to increase the number of chips that are fabricated on a plate, thereby increasing the productivity of the machine. However, the extent to which chip area can be reduced is limited by the resolution of the microprojection system, i.e. by the diameter of the spots, and also by the spacing between spots which depend on numerous factors, such as for example: the microvolume that is projected, the directional stability of projection, the extent to which microdroplets spread, the surface state of the substrate, . . . .

In general, it is possible to design the machine so that spot diameter is of the order of 1 μm to 500 μm, and more particularly lies in the range 200 μm to 300 μm, with spacing between spots generally lying in the range 1.25 to 4 times spot diameter.

As shown in FIG. 8, the volume of reagent 3 is constituted by an available volume 3a, a dead volume 3b situated beneath the outlet tube 12, and a priming volume 3c that goes all the way to the substrate 60 that forms the ejection nozzle 14. The projectable volume of reagent is constituted by the available volume 3a and the priming volume 3c, it being understood that it is also possible to project the dead volume 3b providing it is possible to compensate for the variation in flow rate that occurs when the area of the reagent subjected to gas pressure changes after the available volume 3a has been used up.

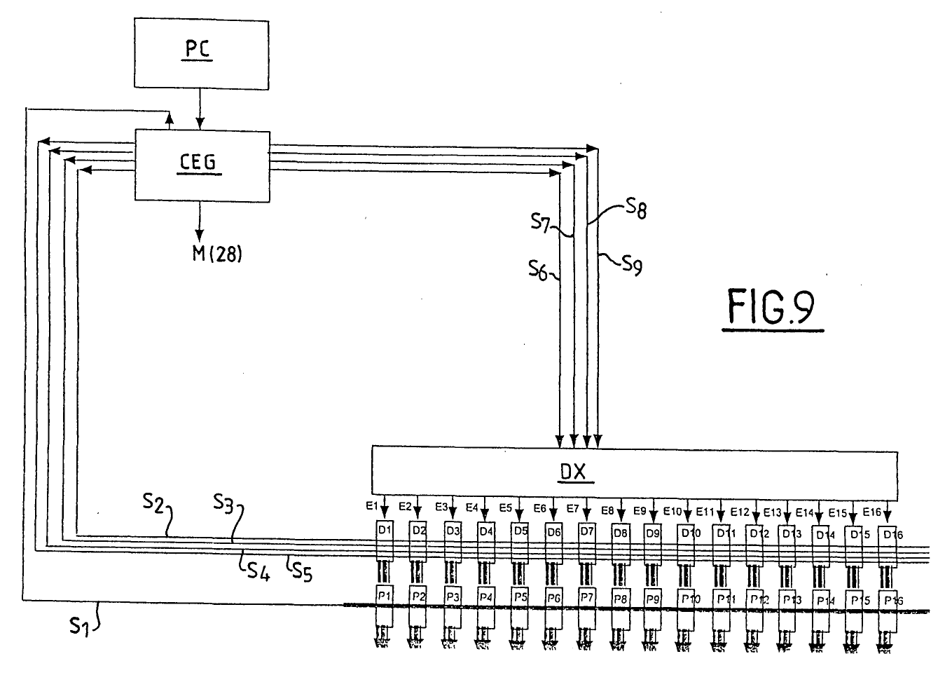

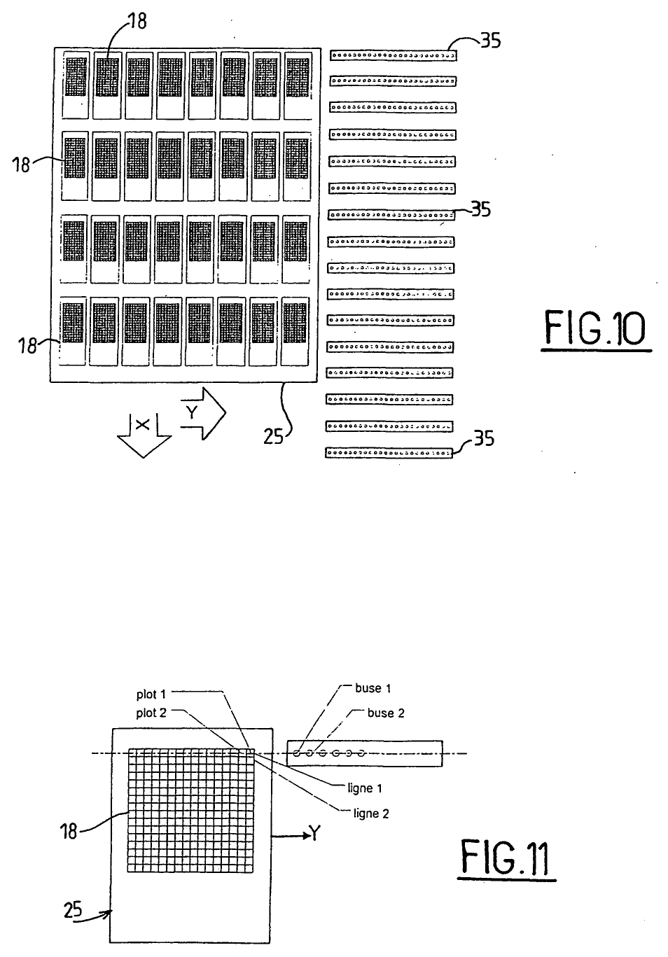

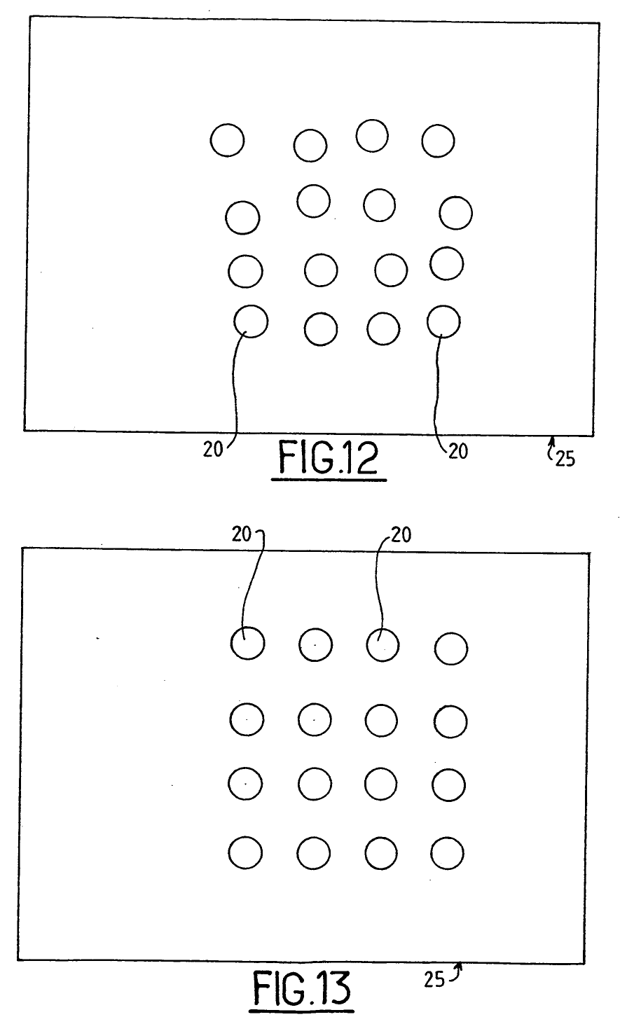

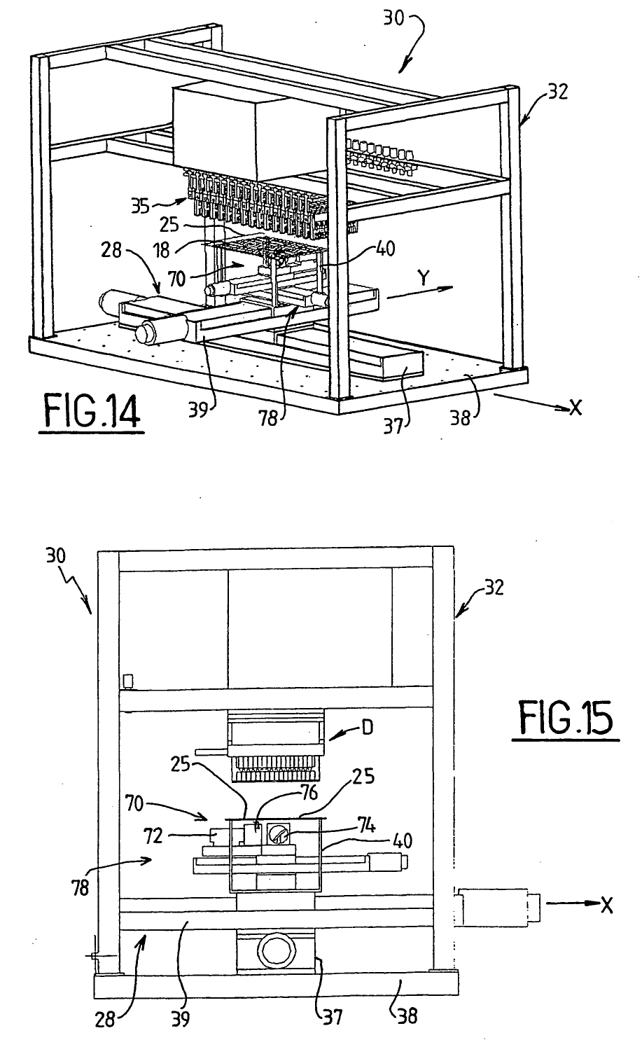

FIG. 9 is in the form of a block diagram showing an embodiment of a control device 30a of the machine 30, the control device comprising a PC associated with a general electronic control unit CEG which governs:
displacements of the plate 25 by suitably controlling the two motors M of the device 28 via two power cards, which take account of the time required for acceleration and deceleration;
synchronization of the X and Y displacements of the plate 25 with firing instructions; and
selecting modules 35 and selecting the valves 22 of the selected modules.

Specifically, the control device 30a controls sixteen modules 35 of rank i (i=1 to 16), each being associated with sixteen microprojection devices of rank j (j=1 to 16), giving a total of 256 microprojection devices (i,j).

In order to control these 256 microprojection devices, various electronic devices are known to the person skilled in the art. By way of example, a control device is described based on the following principles:
one power circuit 22a (FIG. 2) per microprojection means, giving a total of 256 control transistors;
a trigger control that is common to all 256 power transistors:
a specific selection signal for each transistor; and
2-stage demultiplexing making it possible to select:
   a module of rank i; and
   the 16 microprojection means having the same rank j in the set of modules, i.e. (1,j) to (16,j), thereby making it possible to actuate the signal for selecting transistor (i,j) at the intersection between these two selections.

Specifically, each module 35 is connected to an electronic control card which is split into two portions: a logic portion Dᵢ and a power portion Pᵢ.

The logic portion Pᵢ is constituted by a 4-input demultiplexer, whose inputs are respectively connected to four control outputs S₂, S₃, S₄, and S₅ of the card CEG, the demultiplexer having sixteen outputs which are connected respectively to corresponding ones of the inputs of sixteen 2-input AND gates (ij) (not shown), the outputs of the AND gates being connected to the bases of the 16 power transistors of the power portion Pᵢ, the 16 AND gates thus constituting the circuits for selecting the power transistors.

The power portion Pᵢ is subdivided into sixteen control circuits 22a each for controlling one micro solenoid valve 22 and each comprising one transistor T whose base is connected to the output of the corresponding AND gate (ij).

The other input of each AND gate (ij) is connected to a common control output S₁ of the card CEG, thereby constituting the common firing trigger control (which is particularly advantageous for adjusting the length of time the microprojection means remain open).

Furthermore, each demultiplexer Dᵢ presents a disable input Eᵢ, and the sixteen inputs Eᵢ of the demultiplexers D₁ to D₁₆ are respectively connected to sixteen outputs of a demultiplexer DX having four inputs and sixteen outputs. The four inputs of the demultiplexer DX are connected respectively to four control outputs S₆, S₇, S₈, and S₉ of the card CEG.

Thus, in order to control one of the 256 microprojection devices of the machine 30, the card CEG under the control of the PC:
selects one of the 16 modules 35 of the machine 30 via the four control outputs S₆ to S₉ and the demultiplexer DX, thereby enabling one of the 16 demultiplexers Dᵢ;
acts via the four outputs S₂ to S₅ to select that one of the sixteen power transistors of the module 35ᵢ corresponding to the rank j of the microprojection device that is to be activated;
thereby activates a first input of the AND gate (ij); and
sends a firing control signal on its control output S₁ to the second inputs of all the AND gates, thereby having the effect of activating the selected microprojection device whose AND gate (ij) is the only AND gate to have both of its inputs activated.

Software and electronics serve to achieve synchronization between the displacements of the plate 25 and the projection of drops of reagent. Advantageously, projection is performed by firing on-the-fly so as to reduce stages during which the motors M of the moving device 28 carrying the plate 25 are subjected to acceleration/deceleration. Such firing on-the-fly is particularly advantageous along the Y axis which is parallel to the modules 35. Optionally it is then advantageous to cause the projected microdroplets to dry quickly, advantageously by means of a plate that is hot, thereby reducing any risk of microdroplets of reagent spreading due to the fast displacement of the plate 25.

In order to increase fabrication speed, the machine may process substrates in parallel, for example it may process a matrix of 4×8 microscope slides so as to fabricate 32 chips.

In normal operation, i.e. with the tanks 1 at a pressure of about 0.25 bars of inert gas such as helium, and with the micro solenoid valves 22 being opened for a duration of about 1 ms, the projected reagent drops have a volume of about 10 nl. The machine can then make chips each having a matrix of spots with a diameter of about 100 μm to 400 μm and a spacing between centers of the spots of about 200 μm to 1000 μm.

By way of example, with a spacing of about 10 mm between two ejection nozzles 14 and with the center-to-center spacing between two adjacent spots being about 0.5 mm on a given row of a chip, the plate 25 needs to travel a distance of about 10.5 mm before projecting probes. Thus, if it is assumed that the speed in translation of the plate 25 is about 15 cm/s, the time needed for this movement in translation is about 70 ms, i.e. a duration that is much longer than the length of time the micro solenoid valve 22 is open, which time is about 1 ms.

It is therefore advantageous to investigate the influence of the speed in translation of the plate 25 on the overall speed at which chips can be fabricated.

By way of example, when magnetic linear motors are used it is possible to reach speeds in translation of about 10 m/s. It is found that the time the micro solenoid valves 22 are open has no influence on the operation of the machine so long as the speed in translation of the plate does not reach 1 m/s. About 10 ms are needed to travel the distance between two spots, i.e. 10.5 mm, which duration is approximately one order of magnitude greater than the 1 ms duration that the micro-solenoid valves 2 are open. Thus, the speed of chip fabrication can be improved by increasing the displacement speed of the plate 25 up to a value of about 1 m/s.

An example of fabricating 32 chips is described below with reference to FIGS. 10 and 11.

For simplification purposes, it is assumed that the chips are constituted by 256 spots with one type of probe per spot, and that the machine 30 is fitted with 16 modules. Consideration is also given to moving the plate 25 with a stop between each firing or projection operation in a step-by-step mode, and also to displacement at constant speed with firing being performed on-the-fly along the Y axis.

The operating conditions are thus as follows for fabricating a 16×16 spot chip:
one micro solenoid valve 22 is dedicated to each spot, there being only one spot per probe;
the 32 chips are placed on the plate 25 in batches of eight along the Y axis and in four rows along the X axis;
the ejection nozzles 14 of each module 35 are in alignment relative to the 16 chip spots and oriented along the Y axis; and
the 16 micro solenoid valves 22 of any one module 35 are required to project only onto the 16 spots of the row i of each chip.

Operations take place as follows:
a/ Step a: the plate 25 is positioned along the X axis so that the first row of 16 spots in the first row of the eight first chips is aligned with the row of micro solenoid valves 22 constituting the first module 35, it being understood that ejection nozzle 14ᵢ is to project onto spot i in each of eight chips, the plate 25 moving along the Y axis, and that the method in which the spot nearest to the ejection nozzle in question is calculated amounts to saying that the plate 25 must advance continuously along the Y axis without ever reversing until reaching the end of the eight rows of spots and after projecting onto all of the spots.
b/ Step b: the plate 25 is positioned along the X axis so that the row of 16 micro solenoid valves 22 of the module 35ⱼ are in alignment on the following row j of the eight first chips, with displacement taking place along the Y axis in a manner analogous to step a.
c/ Step c: step b is repeated until the 16^{th} row of the first row of eight chips.
d/ Step d: moving onto the second row of eight chips and repeating steps a, b, and c.
e/ Step e: repeating step d up to the 4^{th} row of eight chips.

The above fabrication process has given a fabrication duration of about 28 minutes when fabricating 32 chips in step-by-step mode with a maximum speed of about 15 cm/s between stops, the mean speed being limited by the time required for accelerating and decelerating the motors M that move the plate 25, and fabrication time is only about 4 minutes using the same plate in on-the-fly firing mode along the Y axis with a constant displacement speed of about 15 cm/s, and with step-by-step mode the X axis.

The fabrication speed in the above-described example is thus about 50 chips per hour in stepper mode, but it can reach 500 chips per hour in firing on-the-fly mode.

However, when faster motors M are used, with on-the-fly firing mode along the Y axis at a speed of 1 m/s, the productivity of the machine can reach 4500 chips/hour.

Furthermore, it is also possible to evaluate the influence of reducing the area of the chips on the productivity of the machine. So far it has been assumed that there is only one chip per substrate, whereas for a chip having 400 spots, the working area is 6 mm×6 mm when the spots have a diameter of 250 μm and the spacing of the spots is 300 μm. Thus, the total area of the chip is 8 mm×8 , mm given the safety margins that are needed in practice. It is therefore possible to envisage making 18 chips (6×3) per substrate, thereby raising the fabrication speed to 81,000 chips per hour, thus demonstrating that the projection method and machine implemented by the invention are well adapted to mass production.

In general, the rate at which reagent 3 is projected by the ejection nozzles 14 can be controlled by controlling the length of time the micro solenoid valves 22 are open, and/or the pressure of the gas inside the tanks 1.

The invention also implements a method of quickly decontaminating the microprojection devices, and a procedure for calibrating and for quality control as described below.

Starting from the above-described machine 30 with 256 microprojection devices, it is possible to mass produce a chip having 1024 probes. For this purpose, it suffices to fit the machine with four devices having 256 microprojection devices mounted in line.

Furthermore, it is very easy to vary the flow rates of the microprojection devices by acting on the gas pressure and/or the duration the micro solenoid valves are open. This makes it possible to calibrate the microvolume of reagent that is projected by each microprojection device so as to compensate for dispersion in valve manufacture, and this can advantageously be done while firing on-the-fly. The increase in rate makes it possible to rinse each microprojection device very effectively, either independently or collectively. In the event of accidental clogging, it is also possible to unclog the microprojection device by increasing the delivery pressure of the micro solenoid valve.

To do that, once the chips have been fabricated, the decontamination process consists in replacing the tanks containing probes with tanks containing a cleaning solvent, e.g. water, and in actuating the micro solenoid valves at a high delivery rate so as to clean all of the microprojection devices.

In general, when precise fabrication is required, it is necessary to take account of certain imperfections in the machine 30 as described above. These imperfections relate in particular to firing dispersion between the various micro solenoid valves 22, to inaccuracy in the alignment of the ejection nozzles 14, to inaccuracy in the assembly of the modules 35, and to dispersion in the positions of the motor-driven device 28 providing crossed movements.

Thus, the sum of all of the above imperfections can lead to chips being made with arrays that are irregular, as shown diagrammatically in FIG. 12 for an array constituted by microprojecting 16 microdroplets of reagent onto a substrate 18.

In order to mitigate these imperfections, the method consists in calibrating the machine 30 prior to launching mass production of chips, so as to obtain good reproducibility of operation and obtain regular arrays of microdroplets or spots as formed after the solvent has been eliminated.

By way of example, this method consists in:
causing the micro solenoid valves 22 to fire onto a transparent intermediate substrate;
identifying the positions of the spots, e.g. by means of a camera disposed beneath the substrate, and identifying the differences between the identified positions and the desired positions of the spots; and
correcting the trajectory of the plate 25 which supports the substrate on the basis of a correction table stored in a memory, so that at the instant of microprojection, the XY offset of the plate will compensate for the impact positions relative to other microdroplets, so as to obtain a regular array as shown in FIG. 13.

This type of correction is possible since, in general, the microprojection operations are performed sequentially, i.e. the micro solenoid valves 22 are fired one after another. However, such correction is difficult to make compatible with operation of the on-the-fly type in XY displacement mode, i.e. when a plurality of micro solenoid valves are fired simultaneously.

Nevertheless, under such circumstances, an advantageous compromise can be reached consisting in:
modifying the design of the modules 35 so as to correct for the inaccuracy of each shot along the X axis, with this being achievable by aligning the valves 22 one by one by mechanical adjustment, e.g. by providing the necessary degrees of freedom in rotation for each of the ejection nozzles;
proceeding with calibration along the Y axis;
operating the machine with firing on-the-fly along the Y axis; and
moving the plate 25 in step-by-step mode along the X axis.

In addition, the microvolume of reagent that is projected by a micro solenoid valve depends in particular on the length of time it is open and/or on the gas pressure exerted on the reagent. However, this microvolume also depends on the mechanical construction of the valve. For a machine having 256 microprojection devices, that can lead to dispersion in the microvolumes that are projected and can lead to arrays of irregular spots being formed.

To solve this problem, a first solution in accordance with the invention consists in classifying micro solenoid valves by projection volume and in making modules with valves all having similar volumes, and then compensating for differences between modules by adjusting gas pressure in each module.

Nevertheless, a more advantageous solution consists in varying the length of time the micro solenoid valves are opened in such a manner as to compensate for the dispersions in the microvolumes they project.

Such a procedure for calibrating the projected microvolumes can be performed prior to launching mass production, e.g. by weighing 1000 microdroplets projected by the same micro solenoid valves in a tank, or in line, and weighing the amount projected by a micro solenoid valve using a quartz microbalance placed on the moving plate. Such a method of calibration is advantageous since it enables the projected microvolume to be verified on a regular basis during chip fabrication, and to detect possible problems, for example accidental blockage, variation in volume due to valve aging, sudden microleaks, etc.

Finally, in the invention, it is also possible to verify whether a microdroplet has indeed been projected by using a display system 70, as shown diagrammatically in FIGS. 14 and 15.

The display system 70 is placed beneath the plate 25 and, for example, comprises a camera 72, a 45° mirror 74, a magnifying zoom lens 76, and a lighting device (not shown) for observing the projection of microdroplets of reagent or the formation of spots on the substrates 18 by transparency and observation from below.

In the embodiment shown in FIGS. 14 and 15, the support 40 of the plate 25 is hollow so as to receive therein the display system 70 which is itself mounted on its own motor-driven device 78 for imparting crossed X and Y movements of the same type as that used for moving the plate 25.

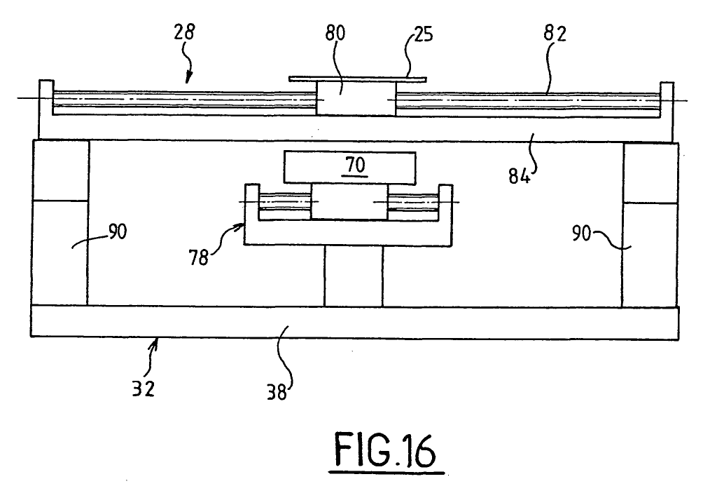

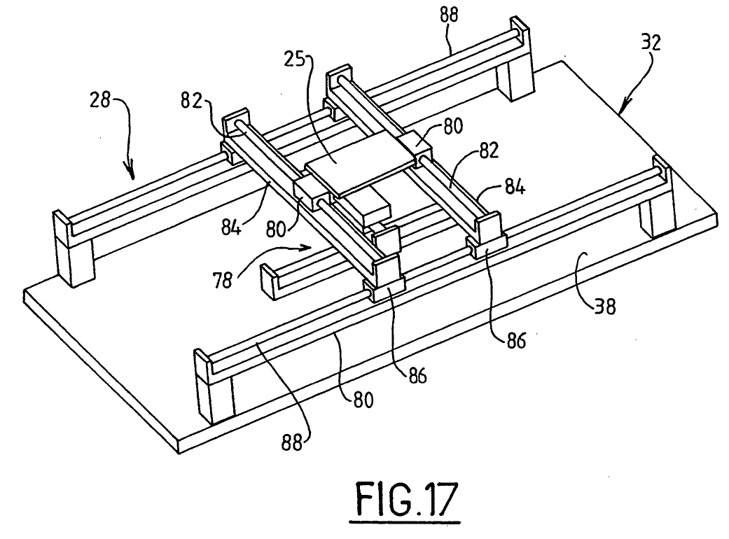

When the travel speed of the plate 25 becomes large, it is advantageous to dissociate the movements of the plate 25 and the movements of the display system 70 so as to distribute masses, and in another embodiment as shown in FIGS. 16 and 17, the display system 70 is not secured to the support 40 but is mounted independently with its own motor-driven device 78. Specifically, it rests directly on the base 38 of the frame 32 of the machine 30.

Furthermore, the motor-driven device 28 for moving the plate 25 is designed differently from that shown in FIG. 4. More precisely, the two opposite sides of the plate 25 are fixed to two first guide blocks 80 passing and driven by two respective first wormscrews 82 which extend parallel to each other. The opposite ends of each wormscrew 82 are supported in rotation by a corresponding strip 84. The two ends of each strip 84 are also secured to two respective second guide blocks 86 passing and driven by two second wormscrews 88 which extend parallel to each other and perpendicularly to the first wormscrews 82. The opposite ends of each second wormscrew 88 are supported in rotation by a corresponding strip 90 which is in turn secured to the base 38 of the structure 32 of the machine 30.

In other words, the plate 25 is mounted on a structure formed by two strips 84 that are movable along the X axis, and said frame is itself mounted to move along the Y axis on a frame formed by two strips 90, which frame is stationary and secured to the structure of the machine 30.

When silicon substrates are used, it is advantageous to provide for the substrates to be polished on both faces so as to be able to make observations by transparency using a camera operating in the near infrared, in association with appropriate lighting.

The machine of the invention comprises a large number of microprojection devices enabling biochips to be mass produced, and such mass production can be performed in a single pass. All of the types of probes that are used can be stored separately in different tanks, thereby avoiding any problem of contamination that might result from changing tanks if the number of biochips to be fabricated were greater than the number of microprojection devices available on the machine.

It can be advantageous to implement the invention together with the following in particular:
the use of functionalized substrates enabling biochips to be obtained that are chemically stable, such substrates being obtainable by methods of silanizing solid supports with self-assembled monolayers (SAMs), as described in French patent application FR-00/00697; and
a chip structure compatible firstly with techniques for detecting nucleic acid hybridization, in particular using methods of marking by radioactive or fluorescent groups, in particular the intercalating group described in French patent application FR-99/11799, and secondly with a method of detecting hybridization as described in French patent application FR-97/07530 which serves in particular to eliminate the stage of marking by means of fluorescent molecules.

The method of manufacture relies on a microprojection system which is adapted to mass producing biochips, and it provides two advantages:
technically: the miniaturization and the simplicity of the system are such that it is possible to envisage putting a thousand microprojection devices together in a battery, each being fed, controlled, and calibrated in totally independent manner; and
economically: the unit price of a complete microprojection device (tank, actuator, tubes, connections, and controlling electronics) is about 150 euros, which means that machines for implementing the method are accessible in terms of cost.

1. A machine for ex situ fabrication of biochips, the machine comprising at least one microprojection device for projecting onto at least one substrate a microvolume of reagent containing at least one probe diluted in an appropriate solvent so as to form, after elimination of the solvent, at least one spot comprising said probe attached to the substrate, the machine comprising a battery of independent microprojection devices for fabricating a plurality of biochips, in particular on plane substrates.

2. A machine according to claim 1, wherein each microprojection device comprises:
a tank in which the reagent for projection is stored;
at least one source of gas under pressure put into communication with the tank via an inlet tube;
an actuator connected to the tank via an outlet tube having one end dipping into the tank; and
an ejection nozzle mounted at the outlet of the actuator and communicating directly with the tank when the actuator is an “open” state under the control of a control circuit constituted by a solenoid valve.

3. A machine according to claim 2, wherein all of the tanks of the battery of microprojection devices are put simultaneously into communication with a common source of gas under pressure.

4. A machine according to claim 2, wherein the battery of microprojection devices is configured in a matrix having a plurality of rows.

5. A machine according to claim 2, wherein the ejection nozzle of a microprojection device is constituted by a tube of PTFE.

6. A machine according to claim 2, wherein the ejection nozzle of a microprojection device is constituted by a part pierced by a hole having a diameter of about 10 μm to 100 μm, and connected to the outlet of the actuator via a connection tube.

7. A machine according to claim 6, wherein said part is constituted by a substrate made of sapphire, ruby, or silicon, a part made of ceramic or of stainless steel, for example.

8. A machine according to claim 4, wherein each row of the battery of microprojection devices forms a module of structure comprising at least:
a first support block in the form of a bar for supporting the set of tanks of the module and for providing the fluid flow connections needed for putting the reagent stored in the tank under pressure; and
a second support block for supporting the actuators and the ejection nozzles of the microprojection devices of the module.

9. A machine according to claim 8, wherein the first support block is pierced by a main longitudinal through channel having one end connected to a source of gas under pressure, and is also pierced by a set of secondary transverse channels each opening out into the main channel and into the set of tanks, there being one secondary channel per tank, thereby enabling a single source of gas under pressure to be used for all of the tanks of the module.

10. A machine according to claim 9, wherein the first support block is also pierced by transverse through orifices through which outlet tubes pass connecting the tanks to the actuators, and wherein said orifices are disposed in a staggered configuration.

11. A machine according to claim 10, wherein each outlet tube is formed by two segments which are connected together at a transverse orifice of the first support block by means of a quick coupling.

12. A machine according to claim 8, wherein the two support blocks are connected to each other by fixing means.

13. A machine according to claim 8, wherein the structure of each module is removably mounted on the structure of the machine.

14. A machine according to claim 1, also comprising a support plate supporting at least one substrate, and means for imparting relative displacement between the plate and the battery of microprojection devices.

15. A machine according to claim 14, wherein the battery of microprojection devices is stationary, and wherein the plate is a moving plate controlled by a motor-driven device delivering crossed XY movements by means of two motors.

16. A machine according to claim 14, also comprising a display system for monitoring the projection of microdroplets or the formation of spots on the substrate.

17. A machine according to claim 16, wherein the display system is placed beneath the substrate-carrying plate.

18. A machine according to claim 17, wherein the display system is mounted on a motor-driven device imparting crossed XY movements.

19. A machine according to claim 18, wherein the motor-driven device imparting crossed XY movements is mounted inside a hollow support for the substrate-carrying plate.

20. A machine according to claim 18, wherein the motor-driven device for imparting crossed movements or moving the substrate-carrying plate and the device for moving the display system are mounted independently of each other.

21. A machine according to claim 20, wherein the substrate-carrying plate is mounted on a first frame movable along the X axis, and wherein said first frame is mounted to move along the Y axis by a second frame which is stationary.

22. A machine according to claim 16, wherein the display system comprises a camera, a 45° mirror, a zoom lens, and a lighting device.

23. A method for ex situ fabrication of biochips, the method being of the type that consists in projecting onto at least one substrate carried by a moving plate, a microvolume of a reagent containing at least one probe diluted in a suitable solvent so as to form, after elimination of the solvent, at least one spot comprising said probe attached to the substrate, wherein, in order to make mass production possible, the method consists in using a battery of independent microprojection devices for projecting microdroplets of reagents in a sequential mode or in an on-the-fly firing mode, in projecting microdroplets at a volume of about 10 nl onto plane substrates, the number of microdroplets lying in the range 1 to 10,000, so as to obtain spots having a diameter of about 100 μm to 1000 μm with inter-spot spacing of about 50 μm to 500 μm.

24. A method according to claim 23, consisting in fitting each microprojection device with a tank containing a reagent and an ejection nozzle, in maintaining the tank under pressure from a source of gas under pressure, and in simultaneously putting all of the tanks of the battery of microprojection devices under pressure simultaneously from a single source of gas under pressure.

25. A method according to claim 24, consisting in associating each microprojection device with an actuator interposed between the tank and the ejection nozzle, and in controlling the actuator to occupy an “open” state during a determined length of time so as to put the tank directly into communication with the ejection nozzle, thereby enabling the microvolume of reagent to be projected under drive from the pressure of the gas present in the tank.

26. A method according to claim 25, consisting in varying the microvolume of projected reagents by acting on the pressure of the gas and/or the length of time the actuator is open.

27. A method according to claim 25, consisting in using a micro solenoid valve as the actuator and in controlling the length of time it is open by means of an electronic control device.

28. A method according to claim 23, consisting in associating each microprojection device with a single type of probe.

29. A method according to claim 23, consisting in forming a plurality of spots on at least one substrate in a single pass without changing the tanks containing the probes.

30. A method according to claim 23, consisting, prior to fabricating chips, in performing a calibration operation so as to obtain a regular array of spots on the substrate, said operation consisting in:
projecting spots onto a transparent intermediate substrate;
identifying the positions of the spots formed on the substrate by means of a camera, e.g. placed beneath the substrate;
recording in a memory the differences between the identified positions and the desired positions of the spots; and
automatically correcting relative displacement between the plate and the battery of microprojection devices to compensate for said differences and obtain a regular array of spots when fabricating chips.

31. A method according to claim 23, consisting in performing quality control to verify whether a spot has indeed been formed on the substrate, said quality control consisting in using a display system mounted beneath the substrate-carrying plate so as to view the formation of spots.

32. A method according to claim 31, consisting in causing the plate and the display system to be displaced independently of each other.

33. A method according to claim 23, wherein, after fabricating chips, a decontamination procedure is performed which consists in replacing the reagent tanks with tanks containing a cleaning solvent, e.g. water, and in actuating the microprojection devices at a high flow rate in order to clean all of the microprojection devices.
